(19) United States
(12) Patent Application Publication
(10) Pub. No.: US 2022/0088365 A1
(43) Pub. Date: Mar. 24, 2022

(54) AUTOLOGOUS BLOOD COLLECTION RESERVOIR

(71) Applicants: Paul Matlin, Salt Lake City, UT (US); Stephen H. McKellar, Salt Lake City, UT (US)

(72) Inventors: Paul Matlin, Salt Lake City, UT (US); Stephen H. McKellar, Salt Lake City, UT (US)

(21) Appl. No.: 17/481,295

(22) Filed: Sep. 21, 2021

Related U.S. Application Data
(60) Provisional application No. 63/081,578, filed on Sep. 22, 2020.

(51) Int. Cl. A61M 60/104; A01N 1/02; A61M 60/35; A61M 60/38

(52) U.S. Cl. CPC A61M 60/104; A01N 1/0247; A61M 2210/125; A61M 60/38; A61M 60/35

(57) ABSTRACT

An autologous perfusion fluid collection reservoir basin includes a basin body configured to receive an extracorporeal organ and blood and includes a sloped bottom. The sloped bottom is configured to channel fluid toward an outlet port. The autologous perfusion fluid collection reservoir basin also includes a connector that extends from the outlet port. The connector is configured to connect to tubing of a cardiopulmonary bypass system or to enable direct connection to a cardiopulmonary bypass system.

### CROSS-REFERENCE TO RELATED APPLICATIONS

This application claims priority to and the benefit of U.S. Provisional Patent Application No. 63/081,578, filed Sep. 22, 2020, the entirety of which is incorporated herein by this reference.

### BACKGROUND

A cardiopulmonary bypass (CPB) system (sometimes referred to as a “heart-lung machine”) includes at least a pump and an oxygenator that may temporarily take over the function of a patient's heart and lungs during surgery. CPB enables various operations, such as, for example, coronary artery bypass surgery, cardiac valve repair/replacement, repair of septal defects, repair of heart defects and/or aneurysms, pulmonary thromboendarterectomy and/or thrombectomy, transplantation (e.g., heart, lung, and/or liver), and/or others.

CPB techniques are costly to perform, carry a great level of risk, and can give rise to various complications for patients. Such cost, risks, and/or complications may be compounded when operations performed using CPB result in patient blood loss, necessitating blood volume replacement. For example, replacing lost blood volume with donated blood may introduce well-known risks associated with donated blood transfusions and may increase the cost of the procedure. In another example, replacing lost blood volume with crystalloid fluids may dilute the patient's blood and may require re-optimization of blood parameters (e.g., optimizing hematocrit), further complicating the procedure and subsequent patient recovery.

Accordingly, there are a number of difficulties associated with CPB-related techniques, systems, apparatus, and/or methods that can be addressed.

The subject matter claimed herein is not limited to embodiments that solve any disadvantages or that operate only in environments such as those described above. Rather, this background is only provided to illustrate one exemplary technology area where some embodiments described herein may be practiced.

### SUMMARY

Embodiments of the present disclosure extend to systems, apparatuses, components, and methods associated with an autologous blood collection reservoir.

For example, an autologous perfusion fluid collection reservoir basin includes a basin body configured to receive an extracorporeal organ and includes a sloped bottom. As used herein, a “sloped” bottom refers to an angled plane or a curved surface that generally lowers toward one side relative to another. The term “slanted” bottom may also be used herein and should also be interpreted as allowing for curved and/or planar surfaces that provide an overall sloped or angled contour enabling fluids to move toward an outlet port via the effects of gravity. The sloped bottom is configured to channel fluid toward an outlet port on the sloped bottom. The autologous perfusion fluid collection reservoir basin also includes an integrated tubing connector that extends from the bottom (or side in some instances) of the outlet port. The integrated tubing connector is configured to connect to tubing of a cardiopulmonary bypass system. Additionally, or alternatively, the reservoir basin and CPB system are configured for a direct connection. For example, a dedicated port can enable direct connection via a plug/socket configuration or other suitable attachment mechanism.

In another example, a method for perfusing an extracorporeal organ includes directing perfusion fluid from a main reservoir of a CPB system to an extracorporeal organ positioned within a collection reservoir basin, collecting perfusion fluid that escapes from the extracorporeal organ within the collection reservoir basin, pumping/sucking the collected perfusion fluid from the collection reservoir basin to the main reservoir, and directing at least a portion of the collected perfusion fluid from the main reservoir into a body of a patient. In some embodiments, including those configured for a direct connection between the collection reservoir basin and the CPB system, a method includes using the collection reservoir basin to receive blood and/or other fluids collected from the operative field. For example, this can include blood collected from towels, sponges, and/or other operating room materials. For example, towels and/or sponges used during a procedure can be wrung out into the collection reservoir basin and the blood and/or other fluids can thereby be returned to the CPB system circuit via operation of the reservoir basin.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be apparent to one of ordinary skill in the art from the description or may be learned by the practice of the teachings herein. Features and advantages of embodiments described herein may be realized and obtained by means of the instruments and combinations particularly pointed out in the appended claims. Features of the embodiments described herein will become more fully apparent from the following description and appended claims.

### BRIEF DESCRIPTION OF THE DRAWINGS

To further clarify the above and other features of the embodiments described herein, a more particular description will be rendered by reference to the appended drawings. It is appreciated that these drawings depict only examples of the embodiments described herein and are therefore not to be considered limiting of its scope. The embodiments will be described and explained with additional specificity and detail through the use of the accompanying drawings in which:

FIG. 1 illustrates schematic representations of example components of an autologous perfusion fluid collection reservoir system;

FIG. 2 illustrates an example autologous perfusion fluid collection reservoir basin;

FIG. 3 illustrates example components of a cardiopulmonary bypass system that may include or implement components of an autologous perfusion fluid collection reservoir system;

FIG. 4 illustrates an example flow diagram depicting acts associated with perfusing an extracorporeal organ.

### DETAILED DESCRIPTION

Embodiments of the present disclosure extend to systems, apparatuses, components, and methods associated with an autologous blood collection reservoir.

For example, an autologous perfusion fluid collection reservoir basin includes a basin body configured to receive an extracorporeal organ and includes a sloped bottom. The sloped bottom is configured to channel fluid toward an outlet port integrated with the basin body. The autologous perfusion fluid collection reservoir basin also includes an integrated tubing connector that extends from the bottom of the outlet port and is configured to connect to tubing of a CPB system. The reservoir basin may additionally or alternatively include an attachment mechanism enabling a direct connection to a CPB system.

In another example, a method for perfusing an extracorporeal organ includes directing perfusion fluid from a main reservoir to an extracorporeal organ positioned within a collection reservoir basin, collecting perfusion fluid that escapes from the extracorporeal organ within the collection reservoir basin, pumping the collected perfusion fluid from the collection reservoir basin to the main reservoir, and directing at least a portion of the collected perfusion fluid from the main reservoir into a body of a patient. Additionally, or alternatively, the reservoir basin may be utilized to receive blood collected in the operative field for return to the CPB system. For example, towels and/or sponges and the like may be drained into the reservoir basin.

The embodiments disclosed and claimed herein can enable the performing of operations that rely on CPB in an advantageous manner. For example, transplantation procedures, such as heart transplant procedures, are performed on patients while the patient is connected to a CPB system. For example, a CPB system may include a venous line (e.g., comprising tubing) that runs from a cannula positioned within the patient's heart (e.g., within the right atrium of the patient's heart) to a main reservoir of the CPB system. The main reservoir may receive venous blood from the patient through the venous line. The CPB system may also include a main pump (e.g., a centrifugal or roller pump) that pumps the venous blood from the main reservoir through an oxygenator to oxygenate the patient's blood (e.g., generating arterial blood). The oxygenated/arterial blood may then be returned (e.g., via the main pump) to the patient's body through an arterial line (e.g., comprising tubing) that runs from the oxygenator to another cannula positioned within the patient's heart (e.g., within the ascending aorta of the patient's heart).

The CPB system may include other components, such as a cardioplegia line and cardioplegia pump (e.g., for administering medication to blood received from the main reservoir and/or oxygenator for delivery to the patient's heart), a temperature control system, a main suction pump (e.g., to provide suction at a cardiotomy site), a left ventricle vent line and/or an aortic root vent line (e.g., to vent air and blood from the patient's heart back to the main reservoir), and/or various auxiliary vent lines, suction lines, and/or pumps.

Before implanting a donor heart into a patient connected to a CPB system, surgeons often perfuse the donor heart with perfusion fluid, in particular blood from the recipient patient. Under conventional approaches/techniques, a surgeon (or appointee) places the donor heart within a slush-filled basin and cannulates the donor heart in preparation for administration of the perfusion fluid. A perfusionist may then deliver the perfusion fluid to the donor heart (e.g., using the cardioplegia pump or another pump of the CPB system), and at least a portion of the perfusion fluid may escape/drain from the donor heart into the basin (e.g., mixing with the slush). The surgeon may then transfer the donor heart to the patient's open chest and continue the implantation procedure.

The perfusion fluid that escapes from the donor heart during pre-implantation perfusion and mixes with the slush within the basin is unfit for direct return to the CPB system or the patient's body, at least in part because the perfusion fluid becomes diluted and would therefore cause undesired changes to the perfusion fluid/blood running through the CPB system (e.g., unwanted dilution). The blood/saline mixture collected in the basin is, therefore, typically collected in a cell saver for future processing.

Pre-implantation perfusion of the donor heart may utilize a significant portion of the perfusion fluid available from the CPB system (e.g., about 500 ml). Accordingly, a perfusionist may need to replace the lost perfusion fluid to ensure safe continuation of CPB. A perfusionist may utilize, for example, donated blood or crystalloid fluid to replace the perfusion fluid lost during pre-implantation perfusion of the donor heart. However, utilizing donated blood may introduce risks and cost associated with donated blood transfusions, and utilizing crystalloid fluid may dilute the perfusion fluid running through the CPB system and may therefore require additional optimization by the perfusionist, thereby delaying and/or complicating the procedure.

Accordingly, at least some of the presently disclosed embodiments are directed toward an autologous perfusion fluid collection reservoir system and methods for using components thereof that may enable a direct return of the perfusion fluid used for pre-implantation perfusion of a donor heart (or another extracorporeal organ) to a perfusion fluid circuit of a CPB system. Advantageously, providing systems and methods that enable a direct return of perfusion fluid used for pre-implantation perfusion of an extracorporeal organ may ameliorate the need for a perfusionist to replace lost perfusion fluid (e.g., recipient patient blood) after pre-implantation perfusion procedures, thereby simplifying transplantation procedures and reducing risks and/or cost associated therewith. Further advantageously, at least some of the components of an autologous perfusion fluid collection reservoir system include or are readily implementable with components of existing CPB machines.

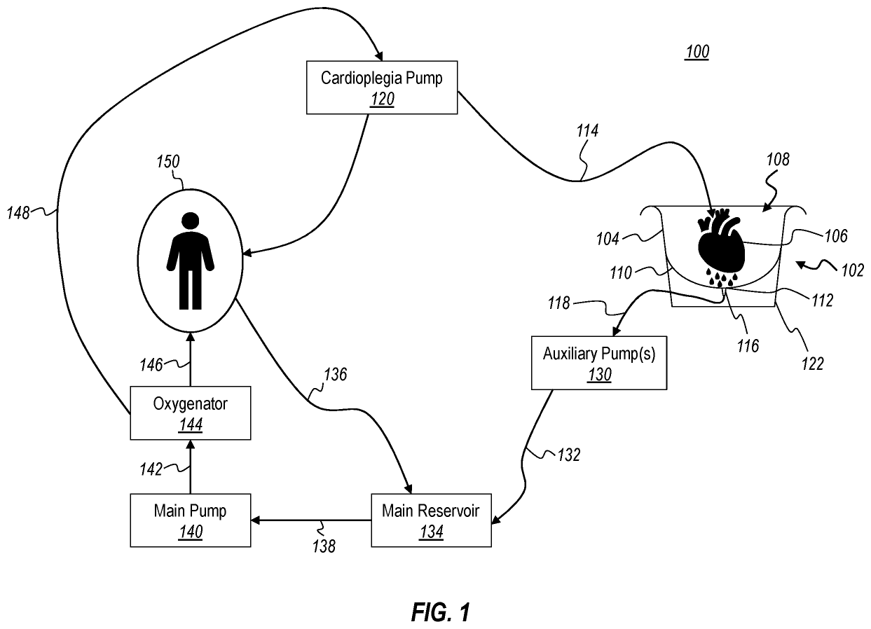

FIG. 1 illustrates schematic representations of example components of an autologous perfusion fluid collection reservoir system 100. The autologous perfusion fluid collection reservoir system 100 illustrated in FIG. 1 comprises a perfusion fluid collection reservoir basin 102 (FIG. 1 provides a cross-sectional representation of the perfusion fluid collection reservoir basin 102). The perfusion fluid collection reservoir basin 102 includes a basin body 104 that is configured to receive an extracorporeal organ, such as donor heart 106. For example, the perfusion fluid collection reservoir basin 102 may include an opening 108 on a top portion thereof. The opening 108 may enable a surgeon (or appointee) to place the donor heart 106 within the perfusion fluid collection reservoir basin 102 and have continued access to the donor heart 106 while the donor heart 106 remains positioned within the perfusion fluid collection reservoir basin 102 (e.g., in preparation for pre-implantation perfusion of the donor heart 106).

Although the present disclosure focuses, in some respects, on heart transplantation, it should be noted that at least some principles disclosed herein can be implemented in other CPB-related procedures (e.g., other transplantation procedures or non-transplantation surgical procedures where blood is to be collected and returned directly to a heart-lung machine).

The implementation of the perfusion fluid collection reservoir basin 102 illustrated in FIG. 1 also includes a sloped bottom 110, which is operable to channel fluid toward an outlet port 112 on the slanted bottom. For example, during pre-implantation perfusion of the donor heart 106, a user (e.g., a surgeon, perfusionist, technician, appointee, or another person) may deliver perfusion fluid (e.g., blood of a patient 150, who is the intended recipient of the donor heart 106) into the donor heart 106 using a cardioplegia pump 120 (or another pump) of a CPB system. A user may cannulate the donor heart 106, position the donor heart within the perfusion fluid collection reservoir basin 102, and connect tubing 114 from the cardioplegia pump 120 to the cannula positioned within the donor heart (in FIG. 1, the arrowheads on the various tubing elements indicate direction of flow). The cardioplegia pump 120 may be connected to other portions of a CPB system to receive perfusion fluid (e.g., the blood of the patient 150). For instance, the cardioplegia pump 120 may receive oxygenated blood from an oxygenator 144 (via tubing 148) that receives blood from a main pump 140 (via tubing 142) that receives blood from a main reservoir 134 (via tubing 138). The user may then deliver any desired amount of perfusion fluid (e.g., blood of the patient 150 circulating through a CPB system) to perfuse the donor heart 106 prior to implantation of the donor heart 106 within the patient 150.

Because the donor heart 106 is unconnected to other systems, the perfusion fluid delivered from the CPB system using the cardioplegia pump 120 may at least partially leak/escape from the donor heart 106, illustrated in FIG. 1 by the drops descending from the donor heart 106 into the perfusion fluid collection reservoir basin 102. Furthermore, the sloped bottom 110 may direct the escaped perfusion fluid toward the outlet port 112 for return to the CPB system through the outlet port 112 (as described in more detail hereinbelow). Because the perfusion fluid collection reservoir basin 102 may facilitate immediate return of the escaped perfusion fluid directly back to the CPB system (connected to the patient 150) that provided the perfusion fluid, the perfusion fluid collection reservoir basin 102 may at least partially ameliorate the loss of perfusion fluid during pre-implantation perfusion of an extracorporeal organ.

Although FIG. 1 illustrates the sloped bottom 110 of the perfusion fluid collection reservoir basin 102 as having an arcuate or concave profile, other slanted, contoured, arcuate, or curved configurations are within the scope of this disclosure (e.g., an arrangement of one or more inclined planes).

FIG. 1 also illustrates that, in some instances, the perfusion fluid collection reservoir basin 102 includes an integrated tubing connector 116 that extends from the bottom of the outlet port 112 below the sloped bottom 110 of the perfusion fluid collection reservoir basin 102. The integrated tubing connector 116 may be sized and shaped to connect to tubing 118, which may be standard tubing for CPB systems/setups. In this regard, at least some perfusion fluid collection reservoir basins 102 of the present disclosure are easily and advantageously implementable with components of existing CPB systems.

The tubing 118 extending from the integrated tubing connector 116 may operate to deliver the perfusion fluid that was collected from the perfusion fluid collection reservoir basin 102 to other portions of the CPB system for eventual return to the patient 150. For example, the perfusion fluid collection reservoir basin 102 may define/include a support wall 122. The support wall 122 may extend downward from the basin body 104 and define at least a portion of the outer perimeter of the perfusion fluid collection reservoir basin 102. The support wall 122 may also form at least part of a flat bottom portion of the perfusion fluid collection reservoir basin 102 below the outlet port 112 and integrated tubing connector 116 thereof, allowing the perfusion fluid collection reservoir basin 102 to freely rest on flat surfaces while still providing the channeling/draining functionality described hereinabove. In this regard, the support wall 122 may enable the perfusion fluid collection reservoir basin 102 to be readily deployed within most operating rooms without specialized stands and/or other equipment. Furthermore, as shown in FIG. 1, the perfusion fluid collection reservoir basin 102 may include a lip on a top portion thereof to facilitate easy handling/positioning of the perfusion fluid collection reservoir basin 102 within an operating room.

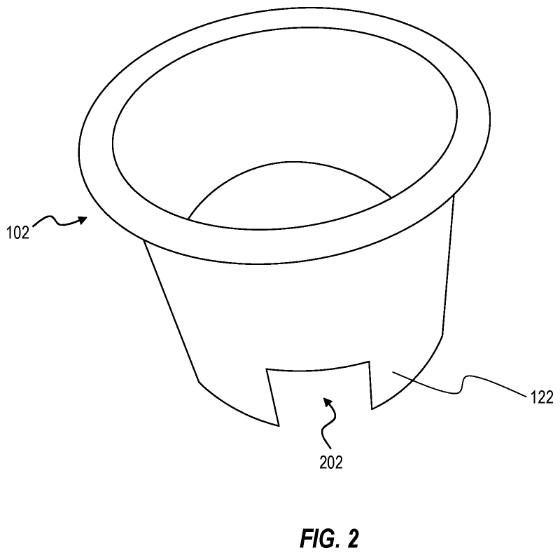

FIG. 2 illustrates an example embodiment of a perfusion fluid collection reservoir basin 102. FIG. 2 illustrates that, in some instances, the support wall 122 defines a wall opening 202. The wall opening 202 may take on various forms and/or shapes, and the implementation illustrated in FIG. 2 is provided by way of example only. The wall opening 202 may provide a path for the tubing 118 that is connected to the integrated tubing connector 116 below the sloped bottom 110 of the perfusion fluid collection reservoir basin. Thus, in some implementations, the wall opening 202 allows the tubing 118 to extend from the integrated tubing connector 116 to deliver collected perfusion fluid to other components of the CPB system while the perfusion fluid collection reservoir basin 102 rests on a flat surface supported by the support wall 122. In other embodiments, an outlet port is disposed at or near the periphery of the support wall 122 and therefore a wall opening 202 may be omitted.

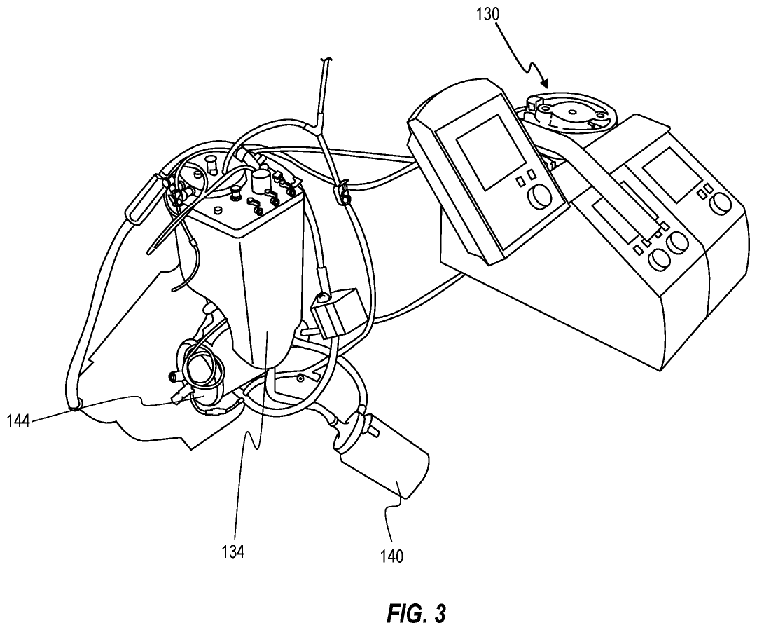

Referring again to FIG. 1, the tubing extending from the integrated tubing connector 116 may run through the wall opening 202 and toward an auxiliary pump 130 of a CPB system (e.g., a roller pump or a centrifugal pump; see, e.g., FIG. 3 which illustrates example auxiliary pumps 130 of a CPB system implemented as roller pumps). The auxiliary pump 130 may pump/direct the perfusion fluid collected within the perfusion fluid collection reservoir basin 102 (e.g., during pre-implantation perfusion of the donor heart 106) to a main reservoir 134 via tubing 132. In this regard, the main reservoir 134 may be configured to receive perfusion fluid collected from the perfusion fluid collection reservoir basin 102 and that passes through the outlet port 112 thereof.

In some instances, the main reservoir 134 is implemented as a venous blood reservoir of a CPB system configured to receive venous blood (e.g., deoxygenated blood) from the patient 150 via tubing 136 (FIG. 3 illustrates an example main reservoir 134 implemented as a venous blood reservoir of a CPB system). Accordingly, the perfusion fluid collection reservoir basin 102 may be configured to provide collected perfusion fluid to the same reservoir (i.e., the main reservoir 134) that receives venous blood from the patient 150, thereby reintroducing the collected perfusion fluid into the CPB circuit for immediate processing and return to the patient 150.

For example, the main reservoir 134 may be connected to a main pump 140 via tubing 138. The main pump 140 may be implemented as a centrifugal or roller pump (FIG. 3 illustrates an example main pump 140 implemented as a centrifugal pump of a CPB system). In some instances, the main pump 140 is configured to replace the function of the heart of the patient 150 during the transplantation procedure (e.g., heart, lung, and/or liver transplantation) or other operations. Thus, the main pump 140 may be configured to draw perfusion fluid from the main reservoir 134 received from the patient 150 via tubing 136 and/or received from the auxiliary pump 130 via tubing 132. The main pump 140 may pump the perfusion fluid from the main reservoir 134 to an oxygenator 144 via tubing 142, and to the body of the patient 150 via tubing 146.

The oxygenator 144 may replace the function of the lungs of the patient 150 during transplantation or other operations (FIG. 3 illustrates an example oxygenator 144 of a CPB system). For example, the oxygenator 144 may oxygenate venous blood (e.g., deoxygenated blood) received from the main pump 140 via tubing 142, thereby generating arterial blood (e.g., oxygenated blood). The arterial blood may then proceed through tubing 146 (being pumped by the main pump 140) to the body of the patient 150 to provide oxygen to the body of the patient 150 while connected to the components of the CPB system. After providing oxygen to the body of the patient 150, the blood pumped through the body of the patient 150 via the main pump 140 may become venous blood (e.g., deoxygenated blood), and the main pump 140 (e.g., in combination with gravity) may further direct this venous blood out of the body of the patient 150 for return to the main reservoir 134 via tubing 136, where tubing 136 may be attached to a cannula in the right atrium of the heart of the patient 150 (or another anatomical structure, such as the super vena cava, the inferior vena cava, etc.). In this regard, the main reservoir may be regarded as an extension of the right atrium of the heart of the patient 150.

Accordingly, an autologous perfusion fluid collection reservoir system 100 may integrate a perfusion fluid collection reservoir basin 102 with components of a CPB system (e.g., a main reservoir 134, auxiliary pump(s) 130, a cardioplegia pump 120, a main pump 140, an oxygenator 144, etc.) to enable direct return (to the CPB circuit that runs through the CPB system and the body of the patient 150) of perfusion fluid used to perfuse an extracorporeal organ (e.g., donor heart 106) prior to implantation of the extracorporeal organ into a patient 150, thereby at least partially ameliorating the need to replace lost volume of perfusion fluid (e.g., with donor blood or crystalloid fluids).

The following discussion refers to a number of acts associated with perfusing an extracorporeal organ (e.g., method acts). The method(s) described below may be carried out utilizing an embodiment as described above. Although the acts may be illustrated or discussed in a certain order as part of one exemplary, preferred embodiment, no particular ordering is required unless specifically stated, or required because an act is dependent on another act being completed prior to the act being performed. Furthermore, it should be noted that, in some instances, at least some of the acts described hereinbelow for perfusing an extracorporeal organ may be omitted or replaced with alternative acts, in accordance with implementations of the present disclosure. In addition, in some instances, additional acts not explicitly described herein may be perform in combination with the acts described hereinbelow for perfusing an extracorporeal organ.

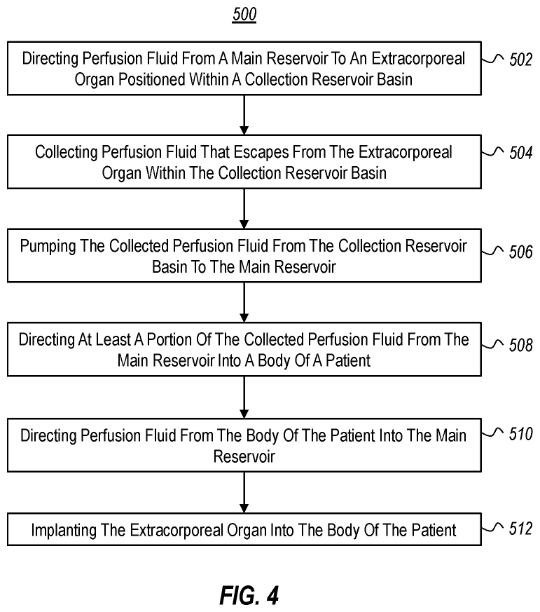

FIG. 4 illustrates an example flow diagram 500 depicting acts associated with perfusing an extracorporeal organ. In particular, flow diagram 500 depicts acts of directing perfusion fluid from a main reservoir to an extracorporeal organ positioned within a collection reservoir basin (act 502), collecting perfusion fluid that escapes from the extracorporeal organ within the collection reservoir basin (act 504), pumping the collected perfusion fluid from the collection reservoir basin to the main reservoir (act 506), directing at least a portion of the collected perfusion fluid from the main reservoir into a body of a patient (act 508), directing perfusion fluid from the body of the patient into the main reservoir (act 510), and implanting the extracorporeal organ into the body of the patient (act 512).

As indicated above, act 502 of flow diagram 500 includes directing perfusion fluid from a main reservoir to an extracorporeal organ positioned within a collection reservoir basin. The collection reservoir basin may correspond to the perfusion fluid collection reservoir basin 102 described hereinabove. Act 502 may be performed as part of a heart transplantation procedure, whereby a user perfuses a donor heart with blood from a patient. The blood from the patient may be drawn from a main reservoir of a CPB system (e.g., a venous blood reservoir), oxygenated (e.g., using a main pump and an oxygenator), and delivered to the donor heart using a cardioplegia pump of the CPB system.

Act 504 of flow diagram 500 includes collecting perfusion fluid that escapes from the extracorporeal organ within the collection reservoir basin. The collection reservoir basin may include a slanted, sloped, or curved bottom that channels the escaped perfusion fluid toward an outlet port on the slanted, sloped, or curved bottom. The outlet port may thereby direct the collected perfusion fluid to other portions of a CPB system for return thereto. Additionally, or alternatively, blood collected from elsewhere in the operative field (e.g., blood in towels and/or sponges) may be directed to the collection reservoir basin.

Act 506 of flow diagram 500 includes pumping the collected perfusion fluid from the collection reservoir basin to the main reservoir. The outlet port described hereinabove with reference to act 504 may include an integrated tubing connector extending therefrom, and the integrated tubing connector may connect to tubing of a CPB system to direct the collected perfusion fluid to the main reservoir of a CPB system. For example, the tubing extending from the integrated tubing connector may run to an auxiliary pump of a CPB system, which may pump the collected perfusion fluid to the main reservoir. The collection reservoir basin may include a support wall that defines a portion of an outer perimeter thereof, and the support wall may include a wall opening to provide a path for the tubing to extend between the integrated tubing connector and the auxiliary pump while the collection reservoir basin rests freely on a flat surface (without resting on the tubing). Alternatively, the collection reservoir basin may include an attachment mechanism that enables direct attachment to the CPB system without the need for tubing.

After return to the main reservoir of the CPB system, the collected perfusion fluid may then advantageously be processed with other perfusion fluid of the CPB circuit without delay. Act 508 of flow diagram 500 includes directing at least a portion of the collected perfusion fluid from the main reservoir into a body of a patient. For example, a main pump may direct the collected perfusion fluid (and other perfusion fluid received from the body of the patient that will receive a donor heart, such as venous blood from the patient) to an oxygenator to oxygenate the collected perfusion fluid. After oxygenation, the main pump may further direct the collected perfusion fluid into the body of the patient.

Act 510 of flow diagram 500 includes directing perfusion fluid from the body of the patient into the main reservoir. For example, the oxygenated collected perfusion fluid described hereinabove with reference to act 508 may provide oxygen to the body of the patient and thereby become deoxygenated perfusion fluid (e.g., venous blood). The main pump may work in concert with gravity to direct the venous blood out of the body of the patient and back into the main reservoir for re-oxygenation and return to the body of the patient.

Act 512 of flow diagram 500 includes implanting the extracorporeal organ into the body of the patient. For example, after perfusing the extracorporeal organ (e.g., a donor heart) with perfusion fluid as described hereinabove with reference to act 502, a user may remove the extracorporeal organ from the collection reservoir basin and bring it to an open cavity of the patient (e.g., the patient's open chest) to implant the extracorporeal organ into the patient. In this regard, at least some of the disclosed embodiments may improve transplantation procedures by enabling users to perfuse an extracorporeal organ with blood from the recipient (or heart-lung machine) and immediately return the blood to a CPB system, thereby avoiding the need to replace lost volume used to perfuse the extracorporeal organ.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. An autologous perfusion fluid collection reservoir basin, comprising:
a basin body configured to receive an extracorporeal organ and comprising a sloped bottom, the sloped bottom being configured to channel fluid toward an outlet port integrated with the basin body; and
a connector extending from the outlet port to enable connection to a cardiopulmonary bypass system.

2. The autologous perfusion fluid collection reservoir basin of claim 1, wherein the connector enables a direct connection of the basin body to a cardiopulmonary bypass system.

3. The autologous perfusion fluid collection reservoir basin of claim 2, wherein the connector includes a plug and/or socket configuration.

4. The autologous perfusion fluid collection reservoir basin of claim 1, wherein the connector is an integrated tubing connector configured to connect to tubing of a cardiopulmonary bypass system.

5. The autologous perfusion fluid collection reservoir basin of claim 1, the basin body further comprising a support wall defining at least a portion of an outer perimeter of the autologous perfusion fluid collection reservoir basin, the support wall defining a wall opening therein.

6. The autologous perfusion fluid collection reservoir basin of claim 5, wherein the wall opening is configured to provide a path for tubing connected to the connector to extend from the connector.

7. The autologous perfusion fluid collection reservoir basin of claim 6, wherein the wall opening is configured to provide a path for tubing connected to the connector to extend therefrom without touching the support wall.

8. An autologous perfusion fluid collection reservoir system, comprising:
a collection reservoir basin configured to receive an extracorporeal organ and shaped to collect perfusion fluid that escapes from the extracorporeal organ, the reservoir basin comprising an outlet port;
a main reservoir configured to receive perfusion fluid that passes through the outlet port of the collection reservoir basin; and
a main pump configured to receive perfusion fluid from the main reservoir, the main pump being configured to direct perfusion fluid into a body of a patient.

9. The autologous perfusion fluid collection reservoir system of claim 8, wherein the collection reservoir basin comprises:
a basin body configured to receive an extracorporeal organ and comprising a sloped bottom, the sloped bottom being configured to channel fluid toward an outlet port integrated with the basin body; and
a connector extending from the outlet port to enable connection to a cardiopulmonary bypass system.

10. The autologous perfusion fluid collection reservoir system of claim 8, wherein the main reservoir comprises a venous blood reservoir of a cardiopulmonary bypass system.

11. The autologous perfusion fluid collection reservoir system of claim 9, wherein the main pump is configured to pump venous blood through an oxygenator for generating arterial blood.

12. The autologous perfusion fluid collection reservoir system of claim 11, wherein the main pump is further configured to pump the arterial blood generated by the oxygenator into a body of a patient.

13. The autologous perfusion fluid collection reservoir system of claim 12, wherein the main pump is further configured to pump the arterial blood through a body of a patient to generate venous blood.

14. The autologous perfusion fluid collection reservoir system of claim 13, wherein the main pump is further configured to direct the venous blood generated by the body of the patient into the main reservoir.

15. The autologous perfusion fluid collection reservoir system of claim 8, wherein the outlet port of the collection reservoir basin comprises a tubing connector configured to connect to tubing to provide perfusion fluid that escapes from the extracorporeal organ to an auxiliary pump of a cardiopulmonary bypass system.

16. The autologous perfusion fluid collection reservoir system of claim 15, wherein the collection reservoir comprises a support wall defining at least a portion of an outer perimeter of the collection reservoir basin, the support wall defining a wall opening therein, the wall opening providing a path for tubing that is configured to extend from the tubing connector of the outlet port of the collection reservoir basin to the auxiliary pump.

17. The autologous perfusion fluid collection reservoir system of claim 8, further comprising a cardioplegia pump configured to deliver perfusion fluid from the main reservoir to a donor heart positioned within the collection reservoir basin.

18. A method for perfusing an extracorporeal organ, comprising:
directing perfusion fluid from a main reservoir to an extracorporeal organ positioned within a collection reservoir basin;
collecting perfusion fluid that escapes from the extracorporeal organ within the collection reservoir basin;
pumping the collected perfusion fluid from the collection reservoir basin to the main reservoir; and
directing at least a portion of the collected perfusion fluid from the main reservoir into a body of a patient.

19. The method of claim 18, wherein the extracorporeal organ is a donor heart and wherein the perfusion fluid and/or the collected perfusion fluid comprises blood of the patient.

20. The method of claim 18, wherein directing the portion of the collected perfusion fluid from the main reservoir into the body of the patient includes oxygenating the portion of the collected perfusion fluid.
